(19) United States
(12) Patent Application Publication
(10) Pub. No.: US 2025/0146605 A1
(43) Pub. Date: May 8, 2025

(54) ASEPTIC TUBING DISCONNECT ASSEMBLY

(71) Applicant: LIFE TECHNOLOGIES CORPORATION, Carlsbad, CA (US)

(72) Inventors: Kim-Xuan Dinh Nguyen, Clear Lake, MN (US); Rick W. Hendrickson, River Heights, UT (US); George J. Vallone, III, DeLand, FL (US); Richard C. Muehling, Azle, TX (US)

(21) Appl. No.: 18/933,336

(22) Filed: Oct. 31, 2024

Related U.S. Application Data
(60) Provisional application No. 63/596,916, filed on Nov. 7, 2023.

(51) Int. Cl. F16L 37/02

(52) U.S. Cl. CPC F16L 37/02

(57) ABSTRACT

Systems and methods for a tubing disconnect assembly are disclosed. In an embodiment the system includes a first bioprocessing unit, a second bioprocessing unit, a flexible tubing in fluid communication with the first and second bioprocessing unit, and a tubular collar having a middle portion sandwiched between a first and second swaged end portions, and secured around at least a portion of the tubing, the collar configured to be disconnected by a disconnecting device in the middle region extending between the first and second swaged end portions.

### CROSS-REFERENCE TO RELATED APPLICATIONS

This application claims the benefit of the U.S. Provisional Patent Application Ser. No. 63/596,916 filed Nov. 7, 2023, and titled “ASEPTIC TUBING DISCONNECT ASSEMBLY,” which is incorporated herein by specific reference.

### FIELD OF THE DISCLOSURE

The present application and disclosure relate to aseptic tubing disconnect assemblies. More specifically, the present application and disclosure relate to systems and methods for aseptic crimping/cutting and sealing of a fluid line or tubing with a mechanically deformable tubing disconnect assembly.

### BACKGROUND

Fluid transfer plays a critical role in bioprocessing operations, which involves the movement of various fluids, such as liquid solutions, media, feed, food, beverage, buffers, and harvested bioproducts, between different bioprocess components. Bioproducts can include medicines, biologics, vaccines, recombinant proteins, monoclonal antibodies, and viral vectors. Maintaining sterility, and preventing cross-contamination, are important during fluid transfer. However, maintaining an aseptic environment for post-fluid transfer steps including disconnection of fluid lines or tubing connected between two or more bioprocess components is also crucial, especially when the bioprocess components are prepared for transport and storage.

Currently, many methods for disconnection are known including, tube sealers, genderless disconnectors, and traditional male-female disconnectors, which can be used to aseptically disconnect a fluid line connecting the bioprocess components. Also known are deformable collar-based disconnections, wherein a collar is disposed around the tubing, and the tubing is disconnected by crimping and/or cutting the collar and tubing, together. For example, at least some currently available collars are single-piece devices with openings, or two-piece devices for ease of applying the collar onto the tubing or are preassembled onto the tubing by adhesives. Single or two-piece collar devices are easily prone to displacement along the tubing while crimping/cutting and adhesive-based collars add complexity and cost to manufacturing. Moreover, some of these collars are limited to certain tubing sizes only. As tubing of different sizes is needed based on the scale of biomanufacturing operations, there exists a need for a more robust, less expensive deformable collars-based tubing disconnect assemblies that can be easily applied to differently sized tubing, and which can provide for easy disconnection to result in an effective aseptic sealing of the disconnected tubing assemblies.

### SUMMARY OF THE DISCLOSURE

It is understood that each independent aspect recited herein may include any of the features, options, and possibilities recited in association with the other independent aspects and dependent aspects set forth above or as recited elsewhere within this document.

Example systems and methods for a tubing disconnect for use in aseptic disconnection of fluid assemblies are herein disclosed. An example tubing disconnect system can include a collar disposed around at least a portion of a flexible tubing, the collar having a first end portion, an opposite second end portion, and a middle axis. A middle portion extends between the first and second end portions, and at least a portion of the first and second end portions can be configured to compressively grip the flexible tubing to avoid movement of the collar on the flexible tubing during the aseptic disconnection of fluid assemblies.

In various embodiments, a method for aseptically disconnecting a fluid line is provided. A first bioprocess component can be setup in fluid communication with a second bioprocess component through the fluid line including a tubular collar secured on at least a portion of the fluid line by a first and second end portions of the tubular collar. An aseptic fluid transfer operation of a fluid can be performed between the first and second bioprocess components through the fluid line. The fluid line can be compressed and disconnected at a first location on the collar to obtain a pair of aseptically disconnected fluid line assemblies. Further the aseptically disconnected fluid line assemblies can be separated.

In various embodiments, a bioprocessing system is provided that includes a first bioprocessing unit, a second bioprocessing unit, a flexible tubing in fluid communication with the first and second bioprocessing units. The bioprocessing system can further include a tubular collar having a middle region sandwiched between a first and second swaged end portions and secured around at least a portion of the tubing. Further, the collar can be configured to be disconnected by a disconnecting device in the middle region extending between the first and second swaged end portions.

### BRIEF DESCRIPTION OF DRAWINGS

FIG. 1 is a schematic view of a bioprocessing system including a tubing disconnect assembly according to exemplary embodiments of the present disclosure.

FIG. 2 is a schematic view of another tubing disconnect assembly according to exemplary embodiments of the present disclosure.

FIG. 3 is a schematic view of a portion of the tubing disconnect assembly shown in FIG. 2 according to exemplary embodiments of the present disclosure.

FIG. 3A to FIG. 3D are cross-sectional views at four different locations on the tubing disconnect assembly shown in FIG. 3 according to exemplary embodiments of the present disclosure.

FIG. 4 is a schematic view of an isolated collar of tubing disconnect assembly shown in FIG. 1 according to exemplary embodiments of the present disclosure.

FIG. 4A is a cross-sectional view of collar shown in FIG. 4 along line A-A according to exemplary embodiments of the present disclosure.

FIG. 5 is a schematic view of tubing disconnect assembly according to exemplary embodiments of the present disclosure.

FIG. 5A is a top view of disconnected portions of tubing disconnect assembly shown in FIG. 5 according to exemplary embodiments of the present disclosure.

FIG. 5B is a side view of disconnected portions of tubing disconnect assembly shown in FIG. 5 according to exemplary embodiments of the present disclosure.

FIG. 5C is an end view of a disconnected portion of tubing disconnect assembly shown in FIG. 5 according to exemplary embodiments of the present disclosure.

FIG. 6 is a graphical representation of pull test results conducted on disconnected portions of tubing disconnect assembly shown in FIG. 1 according to exemplary embodiments of the present disclosure.

FIG. 7 is a schematic view of a tubing disconnect assembly according to exemplary embodiments of the present disclosure.

FIG. 8 is a schematic view of a tubing disconnect assembly according to exemplary embodiments of the present disclosure.

FIG. 9A is an isometric view of a swaging tool according to exemplary embodiments of the present disclosure.

FIG. 9B is an isometric view of a swaging tool according to exemplary embodiments of the present disclosure.

FIG. 9C is an isometric view of a swaging tool according to exemplary embodiments of the present disclosure.

FIG. 10 is a flow diagram of a method for aseptically disconnecting a fluid line in fluid communication between a first bioprocess component and a second bioprocess component in accordance with example embodiments.

FIG. 11 is a flow diagram of a method of manufacturing a tubing disconnect assembly in accordance with example embodiments.

The figures may not be to scale in absolute or comparative terms and are intended to be exemplary. The relative placement of features and elements may have been modified for the purpose of illustrative clarity. Where practical, the same or similar reference numbers denote the same or similar or equivalent structures, features, aspects, or elements, in accordance with one or more embodiments.

### DETAILED DESCRIPTION

Certain embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting embodiments and features illustrated or described in connection with one embodiment may be combined with the features of other embodiments. Such modifications and variations are intended to be included within the scope of the present invention.

Further, in the present disclosure, like-named components of the embodiments generally have similar features, and thus within a particular embodiment, each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

The present disclosure relates to systems and methods including various embodiments of tubing disconnect assemblies that can provide efficient disconnection and effective sealing of fluid lines or tubing or tubing elements in conditions that require minimal contamination of the tubing. Alternatively, or in addition, various embodiments of the tubing disconnect assemblies described herein are applicable to tubing of varied sizes when compared to at least some currently available tubing disconnects, which can allow users to perform fluid transfer with tubing in a variety of conditions and locations, including small to large scale biomanufacturing processes. For example, at least some currently available tubing disconnect assemblies are multi-piece components or use adhesive. As a result, currently, available tubing disconnects are not configured for efficient and effective disconnecting and sealing of differently sized tubing in industrial processes and equipment that require sterile conditions. The exemplary aseptic tubing disconnects of the present disclosure and described below address current shortcomings in a variety of ways. The exemplary aseptic tubing disconnects provide for adhesive-free tubing disconnect assemblies, hence avoiding manufacturing complications involving adhesives. The disconnected tubing assemblies are aseptically sealed and provide for safe storage and transport. The exemplary aseptic disconnects can disconnect and seal tubing associated with equipment that is difficult to access within an industrial process, such as a small to large-scale bioproduction/biomanufacturing process. The exemplary aseptic tubing disconnects can also disconnect and seal multiple types of tubing and tubing elements having different shapes, sizes, materials of construction, inside diameters, and outside diameters.

The example tubing disconnect assemblies disclosed herein can be used to disconnect tubing that contains or is used to flow one or more biological components, fluids, solids, mixtures, solutions and suspensions including, but not limited to, bacteria, fungi, algae, plant cells, animal cells, white blood cells, T-cells, cell media, protozoans, nematodes, plasmids, viral vectors, blood, plasma, organelles, proteins, nucleic acids, lipids, plasmids, carbohydrates, and/or other biological components. The example aseptic disconnects disclosed herein can be used to disconnect tubing used to connect bioprocessing equipment and instruments and flow biological components between bioprocessing equipment and instruments including, but not limited to, connecting one or more reactors, fermenters, centrifuges, centrifugal separators, chromatography units, mixers, homogenizers, magnetic processing units, blood separating devices, filters, bubble traps, motors, pumps (for example peristaltic pumps), scales, agitators, temperature control units, sensors, and/or other bioprocessing equipment and instruments.

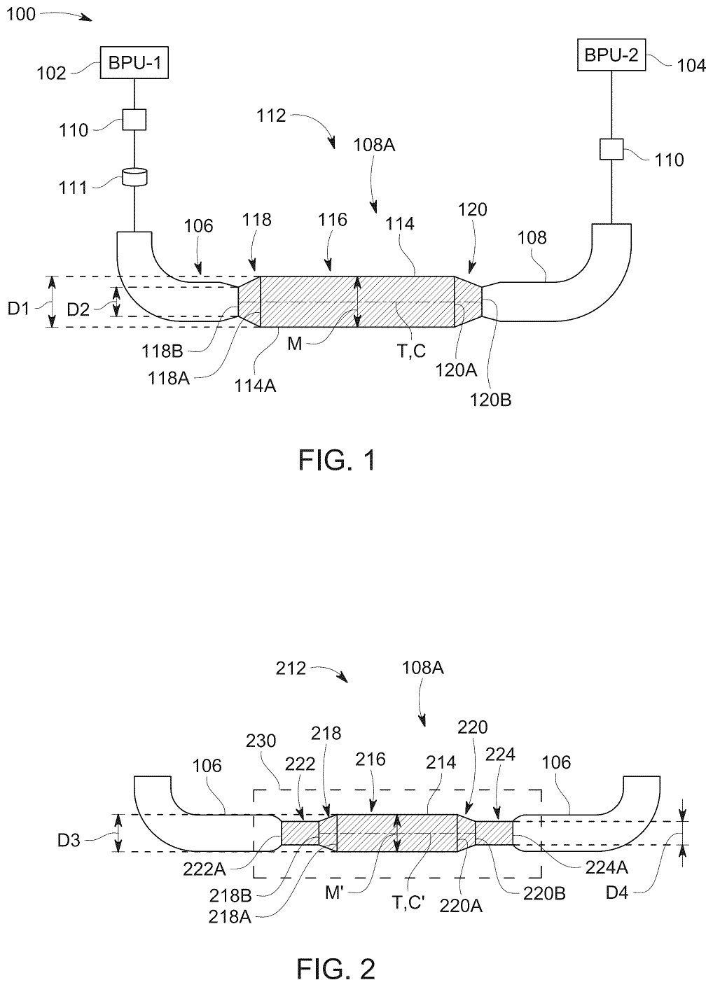

FIG. 1 illustrates an embodiment of a bioprocessing system 100. System 100 includes a first bioprocessing unit 102 in fluid communication with a second bioprocessing unit 104 through a fluid line 106. For example, the first bioprocessing unit 102 can be a fluid source that stores and/or delivers a fluid to be transferred, and the second bioprocessing unit 104 is a receiving container. The first bioprocessing unit 102 can comprise a rigid container, such as a stainless steel container that holds the fluid, or can comprise a flexible bag, such as a closed bag or an open top liner, that can be supported within a rigid support housing or rest on a support surface. The bag can be a two- or three-dimensional bag formed from one or more flexible sheets of polymeric film. The first bioprocessing unit 102 can also comprise a mixing system where the fluid is mixed and/or prepared and can also include a reactor, such as a bioreactor or fermentor. Alternatively, the second bioprocessing unit can be a fluid source and the first bioprocessing unit 102 can be a receiving container.

Fluid line 106 can include flexible tubing 108 or other types of flexible, semi-flexible, semi-rigid conduits that extend from the first bioprocessing unit 102 to the second bioprocessing unit 104. Fluid line 106 allows for fluid flow paths facilitating fluid transfer between first bioprocessing unit 102 and second bioprocessing unit 104. Generally, tubing 108 associated with bioprocessing units 102, 104, is cylindrical in shape and has a circular cross-section, but other cross-sections for tubing 108, including, square, rectangular, and oval, fall under the scope of this disclosure. Tubing 108 can be braided or unbraided and be made of liquid silicone rubber (LSR), thermoplastic elastomer (TPE), polyvinyl chloride (PVC), platinum-cured silicone (PCS), heat cured rubber, or other material suitable for medical or pharmaceutical manufacturing applications. Details of fluids used for fluid transfer through the tubing and the physical conditions the tubing will be exposed to during bioprocessing help in selecting the type of material for tubing 108. Bioprocessing operations use a wide range of sizes for tubing 108, including inner diameter (ID) ranging between 0.125 inches to 0.750 inches, and outer diameter (OD) ranging between 0.250 inches to 1.125 inches. A thickness of tubing 108 is provided by half of the difference between the outer and inner diameters and can range between 0.125 inches to 0.375 inches.

One or more connectors 110 can be used to connect fluid line 106 to the first and second bioprocess containers 102, 104. Connector 110 can comprise a non-aseptic connector that requires coupling in a sterile environment or subsequent sterilization or can comprise an aseptic connector, such as the KLEENPAK Sterile Connector produced by the Pall Corporation, which enables sterile coupling in a non-sterile environment. One or more particulate filters 111 can be disposed of along fluid line 106 so that the fluid passing from fluid source 102 to the second bioprocessing unit 104 passes through particulate filter 111. Particulate filters 111 are non-sterilizing filters that have a size cutoff greater than the size of bacteria. For example, particulate filter 111 can be sized to remove material having a size greater than 300 nm, 0.5-micron, 1 micron, 2 microns, or other sizes. Often, particulate filters 111 are sized to remove material having a size in a range from 0.5 microns to 2 microns. As a result of removing only relatively large particles relative to sterilizing filters, particulate filter 111 permits higher fluid flow rates than when sterilizing filters are used.

Bioprocessing system 100, further comprises a tubing disconnect assembly 112, comprising a collar or sleeve 114 disposed around a portion 108A of the tubing 108. The collar 114 can be disposed of around other portions along tubing 108, between the first and second bioprocessing units 102, 104. Collar 114 can be disposed of proximate to first bioprocessing unit 102 or second bioprocessing unit 104 to facilitate disconnection of the tubing at a desired location along the length of tubing 108. Optionally, one or more collars 114 can be disposed of between first and second bioprocessing units, wherein at least one collar 114 of the one or more collars 114 is proximate to each of first and second bioprocessing units 102, 104. As such, information regarding the further use of the first and second bioprocessing units 102, 104 in the biomanufacturing process can be a factor among others in selecting an appropriate location for the collar 114 on tubing 108.

As shown in FIG. 1, in reference to portion 108A of tubing 108 having a longitudinal axis T, collar or sleeve 114 has a tubular structure and extends lengthwise along a longitudinal axis C, and radially around axis C. In the present example, longitudinal axes T, and C coincide with each other and are orthogonal to a middle axis M. In other words, collar 114 is coaxial with the portion 108A of tubing 108, and both collar 114, and tubing portion 108A have coaxial circular cross sections. Generally, collar 114 and tubing 108 have matching cross-sectional shapes. Further, collar 114 comprises a middle portion 116 sandwiched between a first-end portion 118, and an opposite second-end portion 120. It may be noted that each of middle, first-end, and second-end portions 116, 118, 120 extend along longitudinal axis C. First and second end portions 118, 120 are spaced symmetrically and equally in opposite directions from middle axis M of collar 114. First end portion 118 of collar 114 includes proximal end 118A proximal to middle axis M, and a distal end 118B distal from middle axis M. Similarly, the second end portion 120 of collar 114 includes a corresponding proximal end 120A proximal to middle axis M, and a distal end 120B distal from middle axis M.

Collar 114 comprises an outer wall 114A with a first diameter and an opposite inner wall (not shown in FIG. 1A) with a second diameter. For reference throughout this disclosure, an average of the first diameter and the second diameter of collar 114 is termed as the diameter of collar 114. In the present example, collar 114 has a diameter D1 in middle portion 116, and diameter D1 is equal to outer diameter (OD) of tubing 108. In a preferred embodiment, collar 114 can be made of a metal selected from the group consisting of aluminum, brass, and stainless steel. In other embodiments, collar 114 can be built from other materials having similar deformability characteristics (malleability, pliability, elasticity) to the example metals mentioned above. Collar 114 is configured to deform in response to a first force applied by a compressing device and get cut in response to a second force applied by a disconnecting device. In example embodiments the compressing device and disconnecting device can be a single device or two distinct devices. Upon application of the first force by the compressing device followed by the application of the second force by the disconnecting device at a location on collar 114, tubing 108 enclosed therein is aseptically sealed by the deformed collar. In a preferred embodiment, collar 114 has a length ranging between about 1.0 to about 4.0 inches. Other lengths for collar 214 selected based on the inner and outer diameters of tubing 108 fall under the scope of this disclosure.

It may be noted that first end portion 118, and second end portion 120 are swaged end portions or frustoconical end portions. In other words, middle portion 116 of collar 114 has a diameter D1, and proximal ends 118A, and 120A of first and second end sections also have a diameter D1, but the diameter of collar 114 radially decreases between proximal ends 118A, 120A, and their respective distal ends 118B, 120B. It can also be noted that the diameter of the collar 114 radially decreases continuously from D1 at proximal ends 118A, 120A to D2 at their respective distal ends 118B, 120B. In particular, collar 114 is disposed around portion 108A of tubing such that at least portions of first and second end portions compressively grip tubing 108. In other words, distal ends 118B, 120B of first and second end portions 118, 120, respectively, having a reduced diameter D2 are configured to be compressively received on tubing 108 so as to prevent any displacement of collar 114 along tubing 108 when it is subjected to the first and second forces by compressing and disconnecting devices, respectively, during aseptic disconnection of tubing 108 described later.

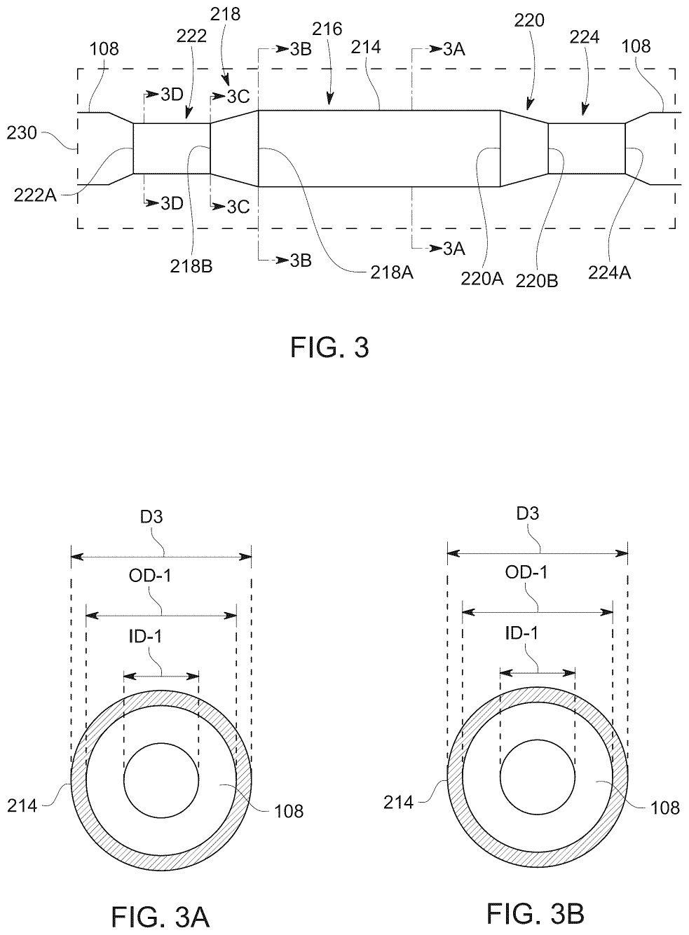

FIG. 2 illustrates an embodiment of another tubing disconnect assembly 212 including a collar 214 disposed to surround tubing portion 108A. Collar 214 is similar to collar 114 and can be used in bioprocessing system 100 as described above. As shown in FIG. 2, in reference to portion 108A of tubing 108 having a longitudinal axis T, collar 214 extends lengthwise along a longitudinal axis C′, and radially around axis C′. In the present example, longitudinal axes T, and C′ coincide with each other, and are orthogonal to a middle axis M′. In other words, collar 214 is coaxial with the portion 108A of tubing 108 and both collar 214, and tubing portion 108A have coaxial circular cross sections. Further, collar 214 comprises a middle portion 216 sandwiched between a first-end portion 218, and an opposite second-end portion 220. It may be noted that each of middle, first-end, and second-end portions 216, 218, 220 extend along longitudinal axis C′. First and second end portions 218, 220 are spaced symmetrically and equally in opposite directions from middle axis M′ of collar 214. First end portion 218 of collar 214 includes proximal end 218A proximal to middle axis M′, and a distal end 218B distal from middle axis M′. Similarly, second end portion 220 of collar 214 includes a corresponding proximal end 220A proximal to middle axis M′, and a distal end 220B distal from middle axis M′.

It may be noted that first end portion 218, and second end portion 220 are swaged end portions or frustoconical end portions. In other words, middle portion 216 of collar 214 has a diameter D3, and proximal ends 218A, 220A of first and second end sections 218, 220, respectively, also have a diameter D3, but the diameter of collar 214 radially decreases between the proximal ends 218A, 220A and their respective distal ends 218B, 220B to diameter D4. It can also be noted that the diameter of collar 214 radially decreases continuously from D3 at proximal ends 218A, 220A to D4 at their respective distal ends 218B, 220B, Additionally, collar 214 includes a first extension portion 222 extending from the distal end 218B of the first end portion 218, and away from middle axis M. A similar second extension portion 224 extends from the distal end 220B of second end portion 220, and away from the middle axis M. Both, first and second extension portions have a reduced diameter D4. In particular, collar 214 is disposed around portion 108A of tubing such that first and second extension portions 222, 224 compressively grip tubing 108. In other words, the first and second extension portions having reduced diameter D4 are configured to be compressively received on tubing 108 so as to prevent any displacement of collar 214 along tubing 108 when it is subjected to first and second forces by compressing and disconnecting devices during aseptic disconnection of tubing 108 described later.

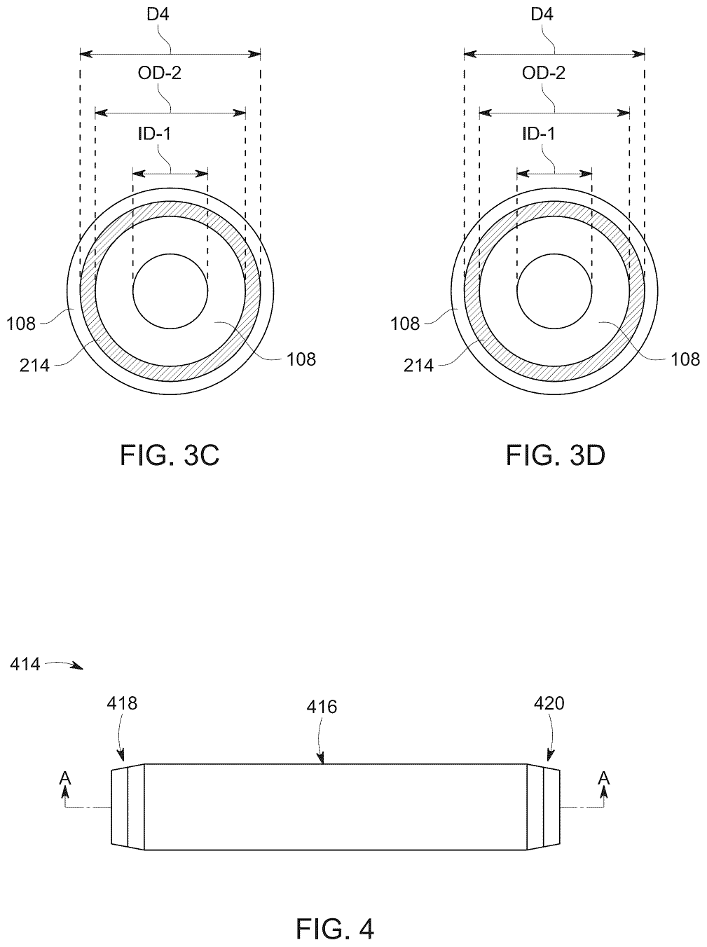

Referring to FIG. 3, a portion 230 of tubing disconnect assembly 212 as shown in FIG. 2 is illustrated. Further, FIG. 3A represents a cross-sectional view along line 3A in middle portion 216 of collar 214. Similarly, FIG. 3B represents a cross-sectional view of collar 214 along line 3B at proximal end 218A of first end portion 218. It can be clearly seen from FIG. 3A, and FIG. 3B, that collar 214 having a diameter D3 is surrounding tubing 108 having inner diameter ID-1 and outer diameter OD-1. FIG. 3C represents a cross-sectional view of collar 214 along line 3C at distal end 218B of first end portion 218, and FIG. 3D represents a cross-sectional view of collar 214 along line 3D at terminal end 222A of first extension portion 222. It can be clearly seen from FIG. 3C, and FIG. 3D, that collar 214 having a reduced diameter D4 is surrounding tubing 108 having inner diameter ID-1 and reduced outer diameter OD-2. Upon receiving first end portion 218 and first extension portion 222 of collar 214, outer diameter OD-1 of tubing 108 is compressed to OD-2 having a reduced diameter. Overall, first and second end portions 218, 220, and first and second extension portions 222, 224 of collar 214 facilitate in securely fixing collar 214 onto tubing 108 to avoid any displacement of collar 214 during the aseptic disconnection of tubing 108 described later. A difference between outer diameter OD-1 and outer diameter OD-2 provides an extent or degree or depth of swaging produced by end portions 218/220 and extension portions 222/224 having swaged structures on tubing 108.

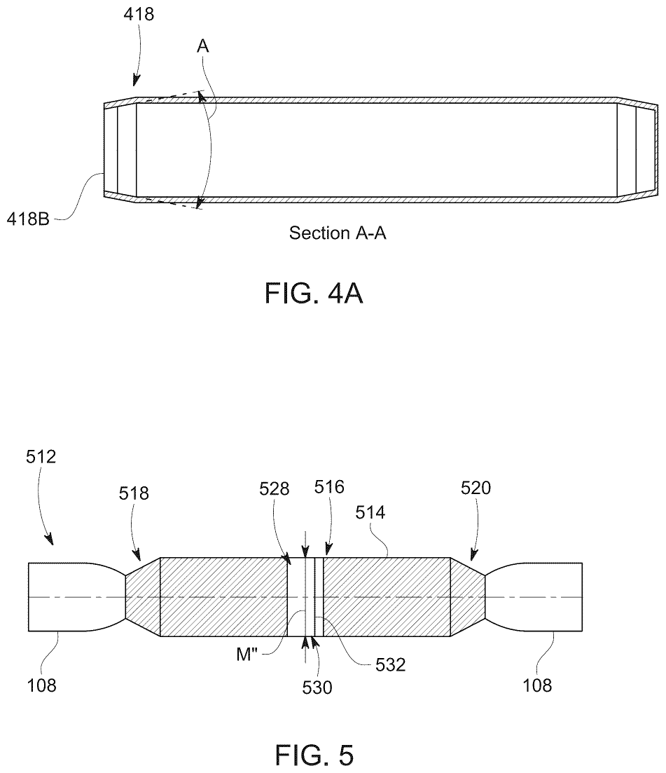

FIG. 4 depicts an embodiment of an isolated collar 414. Collar 414 is similar to collar 114 described in FIG. 1 earlier. Collar 414 includes middle portion 416 sandwiched between first end portion 418, and second end portion 420. In the example depicted in FIG. 4, collar 414 has an overall length of 1.490 inches. FIG. 4A is a cross-sectional view of collar 414 shown in FIG. 4 along line A-A. As shown in FIG. 4A, collar 414 has a diameter of 0.213 inches at a distal end 418B of first end portion 418. An angle A of first end portion having swaged structures is 30 degrees. In other examples, angle A can range between 30-45 degrees. The below Table-1 shows a list of collars 414 with various dimensions. Column-1 lists the sample number, column-2 shows that the diameter of collar can be 22.5 mm or 24.5 mm, and column-3 shows angle A can be 30° or 45°.

TABLE 1

| Sample | D (mm) | A |
|---|---|---|
| 1 | 24.5 | 30° |
| 2 | 22.5 | 30° |
| 3 | 24.5 | 45° |
| 4 | 22.5 | 45° |

In yet other examples, various dimensions for collar 414 are listed below in Table-2. Column 1 lists the sample number, column 2 shows that length (L) can be between 38.1 mm and 101.66 mm, column 3 shows that outer diameter can be between 7.137 mm and 30.988 mm, and column 4 shows that thickness (T) can be between 0.381 mm and 1.27 mm.

TABLE 2

| Sam- | | | | | | |
|---|---|---|---|---|---|---|
| ple | L (Length) | | OD (Outer Diameter) | | T (Thickness) | |
| 1 | 101.66 mm | (4.0″) | 30.988 mm | (1.220″) | 1.270 mm | (0.50″) |
| 2 | 76.2 mm | (2.5″) | 21.209 mm | (0.835″) | 1.016 mm | (0.040″) |
| 3 | 63.5 mm | (2.5″) | 17.780 mm | (0.700″) | 1.016 mm | (0.040″) |
| 4 | 50.8 mm | (2.0″) | 11.049 mm | (0.435″) | 0.762 mm | (0.030″) |
| 5 | 38.1 mm | (1.5″) | 7.137 mm | (0.281″) | 0.381 mm | (0.015″) |

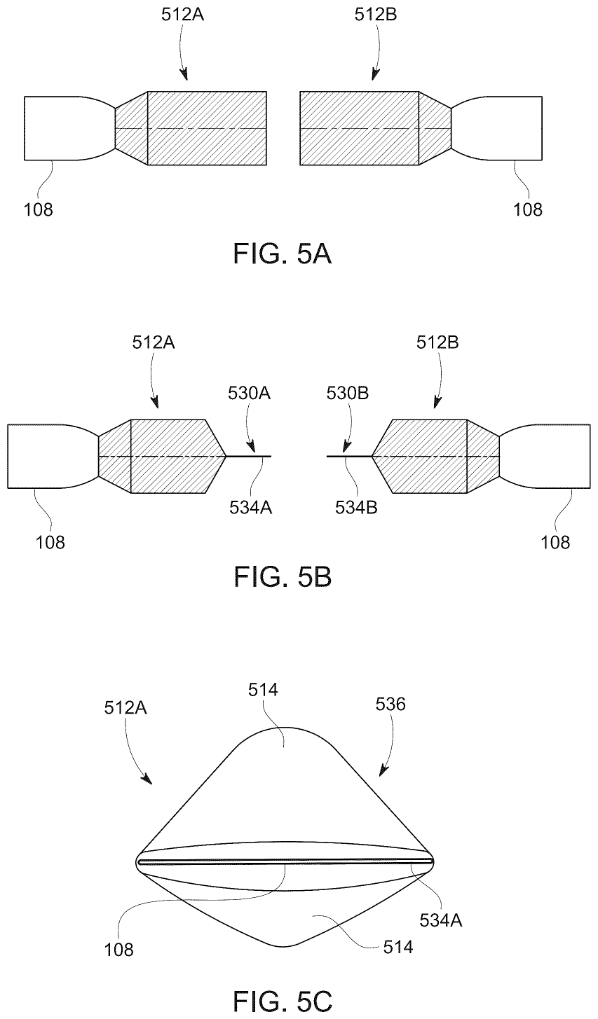

FIG. 5 depicts a tubing disconnect assembly 512 which is similar to tubing disconnect assembly 112 described in FIG. 1 earlier. Tubing disconnect assembly 512 includes a collar 514 secured around tubing 108 by first and second end portions 518, 520 having swaged structures. Tubing disconnect assembly 512 also includes a disconnection region 528 extending across middle axis M″. Firstly, tubing disconnect 512 is subjected to a first force by a compressing device at disconnection region 528 i.e. compressing device is configured to compress both collar 514 and tubing 108 enclosed therein, together, to result in compressed region 530. In practice, tubing disconnect can be subjected to the influence of first force for an extended period of time (for example, 10-30 seconds, 1 min, 2 min, or 3 min) to ensure proper deforming of collar 514 and compression of tubing 108 enclosed therein. In exemplary embodiments, a compressing device can include a crimping device configured to apply a first force to deform collar 512 and compress tubing enclosed therein. Additionally, compressed region can include cut location markings or identifiers 532 in compressed region 528 to facilitate easy identification of an appropriate cut location for tubing disconnect assembly 512. In a preferred embodiment compressed region is located across middle axis M″ and cut locations are located coincident to middle axis M″ or are parallel to middle axis M″ but are required to be located within compressed region 530.

Further, when tubing disconnect assembly 512 is subjected to a second force by a disconnecting device at cut locations 532 in disconnection region 528 or compressed region 530, tubing disconnect assembly 512 is cut/divided into two disconnected assembly portions 512A, 512B as shown in FIG. 5A (top view), FIG. 5B (side view), and FIG. 5C (end view). In exemplary embodiments, disconnecting device can include any device configured to cut, sever, disconnect, separate, splice, divide, break off, detach, tubing disconnect assembly 512 at disconnection region 528 or compressed region 530. As can be seen in FIG. 5B, each of disconnected assembly portions 512A, 512B include their respective compressed portions 530A, 530B, and cut edges 543A, 534B. It is clearly seen in FIG. 5C that cut edge 534A of disconnect assembly portion 512A represents a mechanical seal. Plasticity characteristics of tubing 108 combined with deformable characteristics of collar 514 when subject to a combination of first force by a compressing device followed by a second force by a disconnecting device, efficiently create an effective aseptic seal 536 for each of disconnected assembly portions 512A, 512B. It can also be clearly seen that each disconnected assembly portion 512A, 512B includes a deformed collar aseptically sealing a compressed end of the tubing 108 at a cut edge 534A, and a swaged end portions 518/520 of the collar retaining the deformed collar intact on the tubing 108.

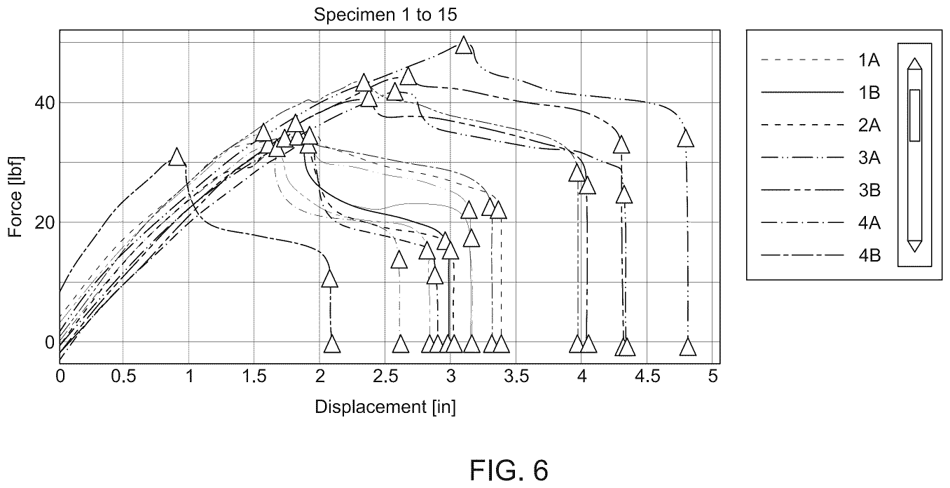

In an exemplary experiment, a pull test was conducted on two disconnected assembly portions 512A, 512B obtained upon disconnecting a tubing disconnect assembly having a collar 514 having a diameter of 1⅛-inch (2.8575 cm) collar-based. Eight pairs of disconnected assembly portions 512A, 512B were fabricated, each pair differing from the other by an angle of swaging A (30 degree or 45 degree) and/or depth of swaging (22.5 mm or 24.5 mm). Each pair of disconnected assembly portions 512A, 512B was checked for a strength by which the deformed collar is retained by their respective swaged end portions 518, 520. An amount of maximum load or load force (lbf) required to pull off the deformed collar from the disconnected tubing portion 512A, 512B shows the efficiency of aseptic seal 536 formed in respective two disconnected assembly portions 512A, 512B. In general, it can be inferred that the more the force required to pull off the deformed portions from the disconnected assembly portions 512A, 512B, the better the aseptic seal 536 and vice versa. Results of the pull test are tabulated in Table 1 below and depicted in a graphical representation in FIG. 6.

TABLE 3

| | | Maximum Load | Angle | Depth |
|---|---|---|---|---|
| S.N | Specimen Label | [lbf] | [degrees] | [mm] |
| 1. | 1A | 32.77 | 30 | 24.5 |
| 2. | 1B | 34.70 | 30 | 24.5 |
| 3. | 2A | 34.41 | 45 | 24.5 |
| 4. | 3A | 49.61 | 45 | 24.5 |
| 5. | 3B | 44.42 | 45 | 22.5 |
| 6. | 4A | 33.15 | 45 | 22.5 |
| 7. | 4B | 31.01 | 30 | 24.5 |
| 8. | 5A | 40.70 | 30 | 24.5 |
| 9. | 5B | 34.76 | 30 | 22.5 |
| 10. | 6A | 34.30 | 30 | 22.5 |
| 11. | 6B | 33.23 | 45 | 24.5 |
| 12. | 7A | 43.63 | 45 | 24.5 |
| 13. | 7B | 35.08 | 45 | 22.5 |
| 14. | 8A | 41.74 | 45 | 22.5 |
| 15. | 8B | 36.84 | 30 | 22.5 |

As can be seen from Table-3 above and the graph in FIG. 6, a mean load required to pull off the deformed collar portion was 37.36 lbf with a standard deviation of 5.36592 lbf. A maximum of 49.61 lbf and a minimum of 31.01 lbf was required for pulling off the deformed collar portion for samples 3A, and 4B respectively. Sample 3A had a swage angle of 45 degrees and sample 4B had a swaging angle of 30 degrees. It may be inferred that a swaging angle between 30-45 degrees provides robust tubing disconnect assemblies.

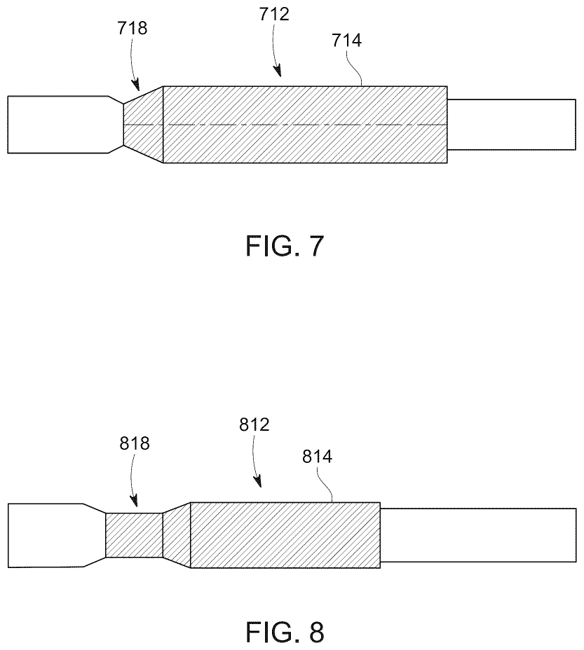

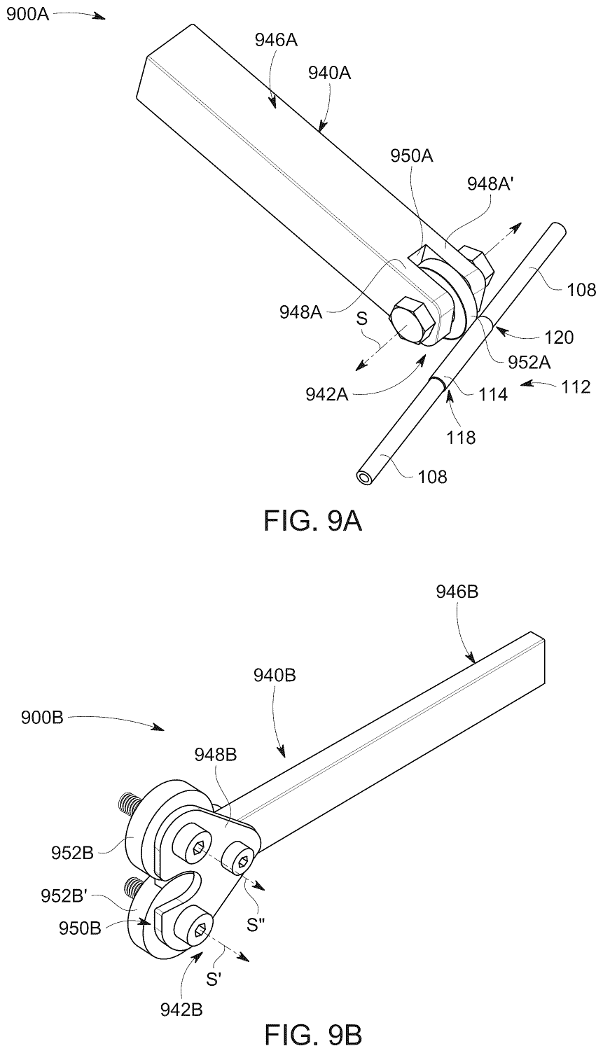

Referring to FIG. 1, and FIG. 2, tubing disconnect assembly 114, illustrated in FIG. 1 has a symmetrical structure as first end portion 118 and second end portion 120 are symmetrically disposed across middle axis M. Similarly, tubing disconnect assembly 214, illustrated in FIG. 2 has a symmetrical structure as first end portion 218, first extension portion 222, and second end portion 220, second extension portion 224 are symmetrically disposed across middle axis M′. Alternatively, tubing disconnect assembly 712 illustrated in FIG. 7 differs from tubing disconnect assembly 114 by being asymmetrical, in having only a first end portion 718 with swaged structure or frustoconical structure at one end of collar 714 and not having a second end portion with swaged structure. Similarly, tubing disconnect assembly 812 illustrated in FIG. 8 differs from tubing disconnect assembly 214 by being asymmetrical, in having only a first end portion 818 with swaged structure, and extension portion 822 at one end of collar 814 and not having a second end portion with swaged structure. Asymmetrical tubing disconnect assemblies 712, 812 can be employed in bioprocessing system 100 described above. In general, when asymmetrical tubing disconnect assembly 712, 812 is connected between a pair of bioprocessing units 102, 104, tubing disconnect assembly 712, 812 can be oriented such that first end portions 718, 818 are proximate to first bioprocessing unit 102, which needs to be aseptically disconnected and distal from the second bioprocessing unit 104 which does not need aseptic disconnection. Examples of such cases can include when bioprocessing unit 102 is a fluid receiver or storage container and all the fluid in second bioprocessing container 104 is transferred to first bioprocessing unit 102 by a fluid transfer process and second bioprocessing unit 104 is prepared for disposal and hence does not need aseptic sealing. Aseptic tubing disconnect assemblies 712, 812 provide for selective aseptic disconnection of a bioprocessing unit 102 in a pair of bioprocessing units 102, 104. Also, manufacturing of aseptic tubing disconnect assemblies 712, 812 having only one swaged end portion would be cheaper and less laborious compared to corresponding tubing disconnect assemblies 112, 212 having two swaged end portions.

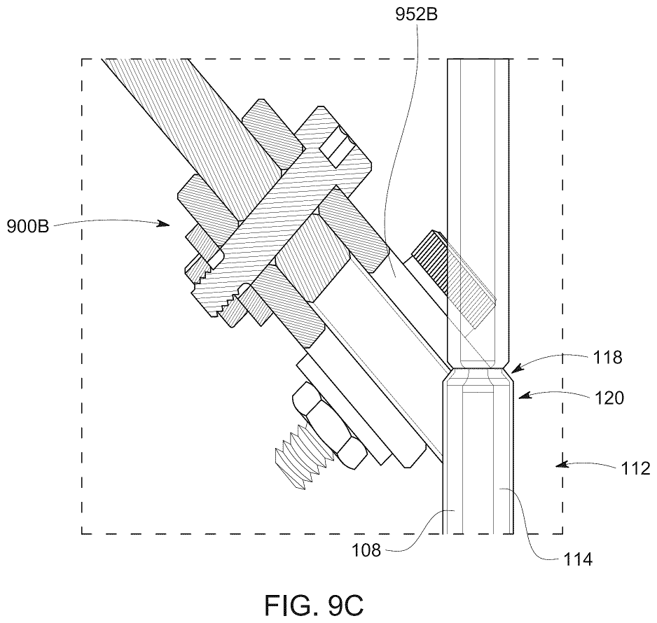

A swaging tool 900A which can be used in producing swaged end portions 118, 120, for collar 114, and swaged end portions 218, 220, and extension portion for collar 214, is as shown in FIG. 9A. Swaging tool 900A includes a body 940A having a head portion 942A and a base portion 946A. Head portion 942A includes a pair of wall structures 948A, 948A′ spaced apart across a passageway 950A. A roller structure 952A located in between wall structures 948A, 948A′ in passageway 950 is held in place by a shaft structure 954 (not shown in FIG. 9). Shaft structure 954 is aligned orthogonal to wall structures 948A, 948A′ and extends through a collinear aperture in each of the opposite wall structures 948A, 948A′. Roller structure 952A is configured for rotation around a central axis S of shaft structure 954. Upon engaging roller structure 952 with a primitive collar disposed on tubing 108, and rotating roller structures 952, swaged end portions 118, 120 can be formed on the primitive collar to result in tubing disconnect assembly 112. Preferably swaging tool 900A is engaged with the collar at an angle of about 90 degrees to produce the swage structures Optionally either swaging tool 900A or collar disposed around tubing 108 can be coupled to a lathe machine configured for rotating the workpiece around an axis of rotation to achieve the swaged end portions on the collar. As previously described in FIG. 4A, angle A of the swaged structure and extent or degree or depth of swaging can be defined based on the size of roller structure, speed of rotation of roller structure 952 and point/location of engagement of roller structures 952 with the primitive collar. For example, roller structure 952 can be a standard ball bearing.

A swaging tool 900B as shown in FIG. 9B, is similar to swaging tool 900A, and can be used in producing swaged end portions 118 for collar 114, and swaged end portions and extension portion for collar. Swaging tool 900B includes a body 940B having a head portion 942B and a base portion 946B. Head portion 942B includes a pair of plate structures 952B, 952B′ spaced apart across a passageway 950B. A pair of roller structure 952B located in between plate structures 952B, 952B′ in passageway 950B is held in place by fastening devices. Each of roller structure 952B is configured for rotation around its respective central axes S′, S″. Upon engaging the pair of roller structure 952B with a primitive collar disposed on tubing 108 as shown in FIG. 9C, and rotating pair of roller structures 952B, swaged end portions 118 can be formed on the primitive collar to result in tubing disconnect assembly 112. Preferably swaging tool 900B is engaged with the collar at an angle of about 45 degrees to produce the swage structures. Optionally either swaging tool 900B or collar disposed around tubing 108 can be coupled to a lathe machine configured for rotating the workpiece around an axis of rotation to achieve the swaged end portions on the collar. As previously described in FIG. 4A, angle A of the swaged structure and extent or degree or depth of swaging can be defined based on the size of roller structure 952B, speed of rotation of roller structures 952B and point/location of engagement of roller structures 952B with the primitive collar. Alternatively, any other tooling that can produce a swaged structure on collars 114 disposed of on tubing 108 can be used.

In example embodiments, a two or four-piece die press having a complimentary shape for forming swaged end portions on collar 114 can be used as a swaging tool. Preferably each piece of the two-piece die set can be engaged with the collar at 90 degrees relative to the collar and each piece of the four-piece die set can be engaged at 45 degrees relative to the collar.

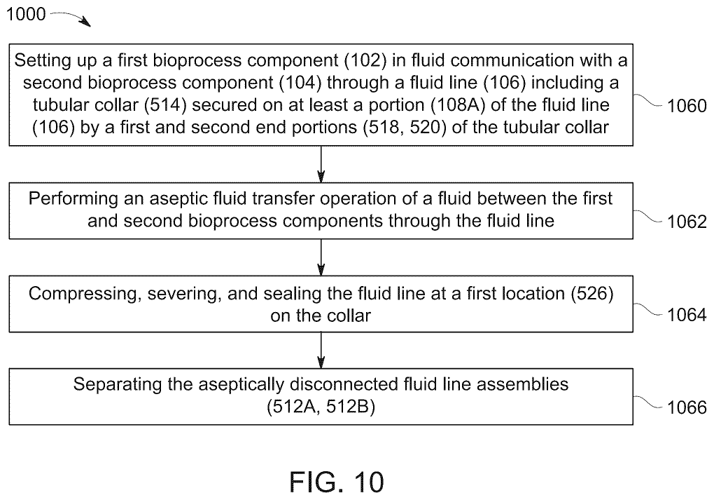

FIG. 10 is a flow diagram of an exemplary embodiment of a method 1000 for aseptically disconnecting a fluid line 106 in fluid communication between a first bioprocess component 102 and a second bioprocess component 104. Aspects of the example tubing disconnect assemblies 112, 212, 512, 612, and 712 as depicted in FIG. 1, FIG. 2, FIG. 5, FIG. 6, and FIG. 7 can be utilized in the method steps described below. These example methods 1000 may not recite the complete process or all steps of the method. Also, the steps need not necessarily all be performed, and in some cases can be performed simultaneously or in a different order than the order shown.

At step 1060, first bioprocess component 102 is set up in fluid communication with second bioprocess component 104 through fluid line 106 including a tubular collar 514 disposed on at least a portion 108A of fluid line 108A by a first and second end portions 518, 520 of tubular collar 514.

At step 1062, an aseptic fluid transfer operation of a fluid is performed between first and second bioprocess components 102, 104 through fluid line 106.

At step 1064, fluid line 106 is compressed or crimped, severed, and sealed at a first location 532 on collar 514. Firstly, step 1064 can include checking for confirmation of completion of fluid transfer operation of the fluid between first and second bioprocess components 102, 104 through fluid line 106. Secondly, step 1064 can include identifying a disconnection region 528 on collar 514. Thirdly, step 1064 can include applying a first force on compressing collar 514 at disconnection region 528 with a compressing tool, for example crimper tool to result in compressed region 530 on collar 514. Optionally, the first force can be applied for an extended time (0.1 min-3 mins) to ensure proper deforming of collar 514 and compression of tubing 108 enclosed therein. Fourthly, step 1064 can include identifying a cut location marking 532 on compressed region 532. Lastly, step 1064 can include applying a second force at cut location marking 532 by a disconnecting device, for example cutter, to result in aseptically sealed disconnected tubing assemblies 512A, 512B.

At step 1066. aseptically disconnected fluid line assemblies 512A, 512B are separated and prepared for storage or transport as required by the processing steps of the biomanufacturing.

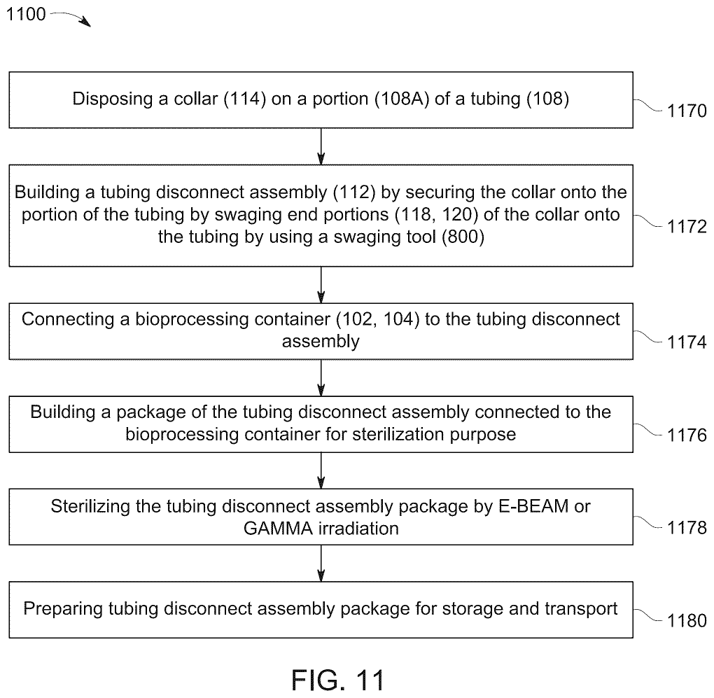

FIG. 11 is a flow diagram of an exemplary embodiment of method 1100 for manufacturing a tubing disconnect assembly 112. Aspects of the example tubing disconnect assemblies 112, 212, 512, 612, 712 as depicted in FIG. 1, FIG. 2, FIG. 5, FIG. 6, and FIG. 7, can be manufactured by the method steps described below. These example methods 1100 may not recite the complete process or all steps of the method. Also, the steps need not necessarily all be performed, and in some cases can be performed simultaneously or in a different order than the order shown.

At step 1170, a collar 114 is disposed on a portion (108A) of a tubing (108).

At step 1172, a tubing disconnect assembly 112 is built by securing the collar onto portion 108A of tubing 108 by swaging end portions of the collar onto the tubing by using a swaging tool 800 at a swaging facility.

At step 1174, tubing disconnect assembly is connected to a bioprocessing unit 102, 104 by using aseptic connectors. For example outlet ports of the bioprocessing units 102, 104 can be configured to connect to tubing 108 by connection retaining devices.

At step 1176, tubing disconnect assembly 112 is connected to a bioprocessing unit 102, 104 and is then packaged for sterilization. Optionally step 1176 can include placing tubing disconnect assembly package in a two-layered bag structure or double bag structure for transfer to a clean room facility for sterilization purposes.

At step 1178, tubing disconnect assembly 112 is sterilized by e-beam or gamma irradiation. Optionally step 1176 can include irradiation in a bulk environment on a pallet (gamma ray irradiation) or a conveyor belt (e-beam irradiation) in accordance with ISO 11137 protocols.

At step 11780, tubing disconnect assembly 112 is prepared for storage and transport.

The different embodiments and examples of the tubing disconnect systems and methods described herein provide several advantages over known solutions for aseptically disconnecting flexible tubing used in the fluid transfer process between two or more bioprocessing components. For example, illustrative embodiments and examples described herein provide for a mechanically robust tubing disconnect assembly suitable for industrial processes that mandate high standards of cleanliness and sterility.

Additionally, and among other benefits, illustrative embodiments and examples described herein provide for tubing disconnect capable of demonstrating visible quality assurance check for confirming the position of the tubing disconnect on the tubing.

Additionally, and among other benefits, illustrative embodiments and examples described herein provide for symmetrical and asymmetrical tubing disconnects.

Additionally, and among other benefits, illustrative embodiments and examples described herein provide for a tubing disconnect including swaged structures for gripping the tubing.

Additionally, and among other benefits, illustrative embodiments and examples described herein provide for cut location identifiers for easily identifying the appropriate location for cutting tubing disconnect assemblies.

Additionally, and among other benefits, illustrative embodiments and examples described herein are configured to allow the user to select a depth or extent or degree and angle of swaging.

Additionally, and among other benefits, illustrative embodiments and examples described herein provide for an effective aseptic sealing of the tubing after the completion of the disconnection process.

### CONCLUSION

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

1. A tubing disconnect, comprising:
a collar disposed around at least a portion of a flexible tubing, the collar having a first end portion, an opposite second end portion, and a middle axis; and
a middle portion extending between the first and second end portions, and at least a portion of the first and second end portions being configured to compressively grip the flexible tubing to avoid movement of the collar on the flexible tubing.

2. The tubing disconnect as recited in claim 1, wherein each of the first and second end portions comprises a proximal end closest to the middle axis and a distal end furthest from the middle axis.

3. The tubing disconnect as recited in claim 1, wherein the first end portion comprises a proximal end closest to the middle axis and a distal end furthest from the central axis.

4. The tubing disconnect as recited in claim 3, wherein a diameter of the collar radially decreases between the proximal end and the distal end of the first end portion.

5. The tubing disconnect as recited in claim 4, wherein the diameter of the collar radially decreases from the proximal end to the distal end of the first end portion.

6. The tubing disconnect as recited in claim 4, wherein the distal end of the collar is compressively received on the flexible tubing.

7. The tubing disconnect as recited in claim 6, further comprising a collar extension portion extending from the distal end and away from the middle axis, and the collar extension portion having a diameter equal to a diameter of the collar at the distal end.

8. The tubing disconnect as recited in claim 7, wherein the collar extension portion is compressively received on the flexible tubing.

9. The tubing disconnect as recited in claim 1, wherein the flexible tubing is in fluid communication between a first bioprocess component and a second bioprocess component.

10. The tubing disconnect as recited in claim 9, wherein the first bioprocess component is a bioreactor and the second bioprocess component is a storage container.

11. The tubing disconnect as recited in claim 9, wherein the first bioprocess component is a fluid source, and the second bioprocess component is a fluid receiver.

12. A method for aseptically disconnecting a fluid line, comprising:
setting up a first bioprocess component in fluid communication with a second bioprocess component through the fluid line including a tubular collar secured on at least a portion of the fluid line by a first and second end portions of the tubular collar;
performing an aseptic fluid transfer operation of a fluid between the first and second bioprocess components through the fluid line;
compressing, and disconnecting the fluid line at a first location on the collar;
obtaining a pair of aseptically disconnected fluid line assemblies; and
separating the aseptically disconnected fluid line assemblies.

13. The method as recited in claim 12, wherein compressing the fluid line at a first location on the collar further comprises applying a first force by a compressing device.

14. The method as recited in claim 13, wherein the compressing device is a crimper.

15. The method as recited in claim 12, wherein disconnecting the fluid line at a first location on the collar further comprises applying a second force by a disconnecting device.

16. The method as recited in claim 15, wherein the disconnecting device is a crimping or cutting tool.

17. The method as recited in claim 12, wherein the first and second end portions have swaged structures.

18. The method as recited in claim 12, wherein the first and second end portions have reduced diameter portions to grip the fluid line.

19. The method as recited in claim 18, wherein the first and second end portions have swaged portions.

20. The method as recited in claim 12, further comprising checking for completion of transferring of fluid between the first bioprocess component and the second bioprocess component prior to compressing, and disconnecting the fluid line at the first location on the collar.

21.-46. (canceled)
